(19) United States
(12) Patent Application Publication
(10) Pub. No.: US 2009/0084379 A1
(43) Pub. Date: Apr. 2, 2009

(54) DRY POWDER INHALER

(75) Inventors: BRUCE A. GOECKNER, Antioch, IL (US); John K. McGeehan, Woodbury, NJ (US); Patrick P. Dayal, Attleboro, MA (US); Kenneth E. Hogeland, Woburn, MA (US); Atif M. Yardimci, Vernon Hills, IL (US); James C. Laird, Grayslake, IL (US); Alice M. Jandrisits, Des Plaines, IL (US); Daniel E. Roush, Niles, IL (US); Craig L. Sandford, Buffalo Grove, IL (US)

Correspondence Address: BAXTER HEALTHCARE CORPORATION, ONE BAXTER PARKWAY, DF2-2E, DEERFIELD, IL, 60015, US

(73) Assignees: BAXTER INTERNATIONAL INC., DEERFIELD, IL (US); BAXTER HEALTHCARE S.A., WALLISELLEN (CH)

(21) Appl. No.: 12/243,342

(22) Filed: Oct. 1, 2008

Related U.S. Application Data
(60) Provisional application No. 60/976,916, filed on Oct. 2, 2007.

(51) Int. Cl. A61M 15/00

(52) U.S. Cl. 128/203.15

(57) ABSTRACT

An dry powder inhaler is disclosed. The dry powder inhaler is designed so that a user's breath vacuums the dry powder from a well within the housing and directs the powder in a direct path to the user. This design prevents impaction and agglomeration of powder within the inhaler. A flow restrictor may be added within the inhaler to increase turbulence and thus increase dispersion of powder within the air inhaled by the user.

### BACKGROUND

The present disclosure relates to inhalers for use in inhaling dry powders. More particularly, the present disclosure relates to inhalers that provide for consistent, uniform lung deposition of an active pharmaceutical ingredient (API) packaged as a dry inhalation powder for systemic lung delivery. The desired lung delivery may be to the local lung or deep lung depending on the API and indication being treated.

Dry powder inhalers or DPI's are one class of devices that are used to deliver medication to a patient through inhalation of the medication into the patient's lungs. Typically, dry powder inhalers include an air flow path or passageway having an inlet and an outlet. A dose of dry powder that is made up of micronized particles is positioned at a location between the inlet and the outlet. A user places his or her mouth at the outlet end of the air passageway and inhales, causing air to enter the inlet end of air passageway and pass through the passageway. As air passes through the passageway, the dry powder is dispersed into the airflow, and exits from the outlet into the patient's mouth and then travels into the lungs along with the inhaled air. Micronized particles generally refer to particles of a size between 0.1 and 10 micrometers, which may be produced by a number of different methods.

Typically, the active pharmaceutical ingredient (API) includes small particles from about 0.1 to about 5 microns in their largest dimension. In certain dry powder forms, these particles have a tendency to agglomerate either by a natural tendency to stick to each other or due to dose packaging that contributes to caking of the micronized powder over time. To minimize the agglomeration and aid in dispersion, these particles are usually combined with respiratory lactose particles, which generally has a size from about 10 to about 120 microns. The small particles containing the drug coat the exterior of the large inert respiratory lactose particles. However, this large particle size does not lend itself to proper transmission to and penetration within the lungs. It is therefore desired to detach or release the small API particles from the larger carrier particles before the combined particles reach the lung and thereby facilitate the delivery of the particles to the lung.

In many dry powder inhalers, dry powder particles are dispersed into the airflow by directing a high velocity stream of air resulting from the patient's inhalation directly onto, along, or through a loaded dose of the powder particles. The air stream will then carry the particles along a tortuous airway where the particles are subjected to turbulent air flow and are also forced to impact on various walls or other impediments, This turbulence and impaction acts to split off or separate the active pharmaceutical ingredient from the lactose carrier. If there is inadequate separation or the combined particles are too large the particles will frequently exit the inhaler into the mouth with such a momentum that they impact on the back of the throat and not even reach the lung.

However, even the particles that have had a large amount of the lactose carrier removed and thereby reach the lung may be of a size, which does not allow deep lung penetration. Also the amount that does reach the lung may be a small portion of the loaded dose, which was placed in the inhaler. Moreover the consistency of the amount of delivered dose may vary widely. The varying of the amount of delivered dose may not deliver the desired therapeutic effect. For certain drugs upper lung penetration may be sufficient to deliver the desired dose and the cost of the drug may be low enough that even a small portion of the beginning dose being actually delivered is still economically feasible.

For other drugs, failure to deliver a consistent dose to the deep lung may not deliver the desired therapeutic effect. In addition the cost of the drug may be so high that it is economically desired to deliver a high percentage of the loaded dose to the lungs. To provide for such delivery one may seek to not use the lactose carrier and instead have the active drug powder without the carrier packaged in the dosage form. However, as noted earlier these particles tend to agglomerate and the inhaler may not be able to properly disperse the agglomerated particles. Agglomerated particles with their larger size have the same drawbacks as the combination carrier ingredient particles discussed earlier. Moreover the stickiness of the particles may cause a portion of the particles to stick to any surfaces in the inhaler that they contact while passing through the inhaler which reduces the dosage that exits the inhaler. Furthermore, in seeking to facilitate dispersion, for certain powdered doses directing the air stream passing through the inhaler into the packaged dose may actually compress the dose and increases the agglomeration of the particles.

In particular it has been found that particles that are composed of protein microspheres, such as insulin, usually have surfaces that tend to adhere to each other and to surfaces that they contact. When such powders are used in present inhalation devices the problems associated with dispersing the agglomerated particles and adhesion to inhaler surfaces are pronounced, and the overall efficiency of the device decreases greatly.

In addition consistency of the delivered dose should be largely independent of patient specific variables of inhalation. For example some patients may be able to apply a much larger negative pressure to the inhalation device than others. In addition patients may vary the amount of negative pressure during the inhalation. Although training may reduce the inconsistency between patients, one can expect this effect to diminish over time and vary among trainers leading back to variations in delivered doses.

Therefore, there remains a need for an inhalation device that facilitates the dispersion of active drug powder and delivers a consistent dose to the deep lung. A related need is to facilitate the dispersion and delivery of a powder made up of micronized particles. A further need is for an inhalation device which tends to reduce the correlation between the patient specific inhalation patterns and variability of delivered dose.

### SUMMARY

The present disclosure is generally related to dry powder inhalation devices that can be used to deliver powder medicaments into the lungs of a user. In particular, the dry powder inhalation device disclosed herein disperses a dosage of therapeutic drug particles which have a natural tendency to agglomerate and minimizes particle collision and impaction on surfaces of the device. This helps in efficiently dispersing powder particles into an air stream within the device, thereby increasing the dosing efficiency of a dose of medication delivered to the patient's lungs. The inhalation devices described herein include an air passageway contoured to create a driven cavity flow so that powder particles are drawn out of a containment reservoir and into the airflow. Additionally, the contour of the air passageway, which may be tapered from both ends to a middle portion of reduced cross-section, reduces the incidence of particle impaction and controls the velocity of the air stream traveling through the passageway. Moreover the contour of the passageway is controlled to create a desired pressure drop through the device for airflows typically created by the inhalation of a user.

In one embodiment, the inhalation device includes a tapering air passageway having an inlet end, a narrow portion, and an outlet end, wherein an inlet cross section of the inlet end is larger than an outlet cross section of the outlet end, and a cross section of the narrow portion is smaller than the inlet and outlet cross sections. The device also includes an air flow restriction between the inlet end and the outlet end, the air flow restriction placed at least partly in the narrow portion of the air passageway, and a well having an opening disposed along the air passageway at the narrow portion and configured to receive a dose of an inhalable powder, the well configured so that a flow of inspired air through the air passageway draws the inhalable powder out of the well and through the inhalation device.

In another embodiment, the inhalation device includes an air passageway having an inlet end, a narrow portion, and an outlet end, wherein an inlet cross section of the inlet end is larger than an outlet cross section of the outlet end, and a cross section of the narrow portion is smaller than the inlet and outlet cross sections, and a well having an opening disposed along the air passageway at or near the narrow portion and configured to receive a dose of an inhalable powder, the well configured so that a flow of inspired air through the air passageway draws the inhalable powder out of the well and through the inhalation device.

Another embodiment of an inhalation device includes a housing having a smooth, single, tapering air passageway having an inlet end, a narrow portion, and an outlet end, wherein an inlet cross section of the inlet end is larger than an outlet cross section of the outlet end, and a cross section of the narrow portion is smaller than the inlet and outlet cross sections. The device also includes a well having an opening that is disposed along the air passageway and configured to receive a dose of an inhalable powder, the well configured so that a flow of inspired air through the air passageway draws the inhalable powder out of the well and through the inhalation device.

Additional features and advantages are described herein, and will be apparent from, the following Detailed Description and the figures.

### BRIEF DESCRIPTION OF THE FIGURES

FIG. 1 is a perspective view of one embodiment of an inhalation device of the present disclosure;

FIG. 2 is a perspective, partial cross-sectional view of the device of FIG. 1;

FIG. 3 is a side cross-sectional view of the device of FIG. 1;

FIG. 4 is a top cross-section view of the inhalation device of FIG. 1;

FIG. 5 is an end, partial cross-section view of the device of FIG. 1;

FIG. 6 is a perspective, partial cross-sectional view of another embodiment of an inhalation device;

FIG. 7A is a side cross-sectional view of the embodiment of FIG. 6;

FIG. 7B is an end, partial cross-sectional view of the embodiment of FIG. 6;

FIG. 8A is an exploded view of another embodiment of the inhalation device of the present disclosure;

FIG. 8B is a perspective view of the dose pack of FIG. 8A;

FIG. 8C is an end view and partial cross-section of the inhalation device of FIG. 8B in the closed or loaded position;

FIG. 9A is an exploded view of another embodiment of an inhalation device the present disclosure shown in an open position with a dose pack;

FIG. 9B is a perspective view, partial cross-sectional view of the inhalation device of FIG. 9A shown in an open position and having the dose pack disposed within the containment well;

FIG. 10A is an exploded view of another embodiment of an inhalation device of the present disclosure;

FIG. 10B is a perspective view of the inhalation device of FIG. 10A shown in the closed or loaded position;

FIG. 10C is an end view and partial cross-section of the embodiment of FIG. 10A;

FIGS. 11A-D are an exploded views and cross-sectional views of another embodiment of an inhalation device of the present disclosure;

FIGS. 12A-D are perspective and cross-sectional views of another embodiment of an inhalation device of the present disclosure; and

FIGS. 13A-c are views of another embodiment of the inhalation device of the present disclosure.

FIG. 14 is a further embodiment of a containment well of the device of FIG. 1.

FIG. 15 is a graph of the flow rates corresponding to various inspiration efforts.

FIG. 16. is a graph of the percentage of particle size deposition in a for a NGI measurement.

FIG. 17 is a bar graph of the emitted dose as a percentage of the loaded dose.

FIG. 18 is a bar graph of the percentage of emitted dose having a size range below 5.0 microns and 3.0 microns.

### DETAILED DESCRIPTION

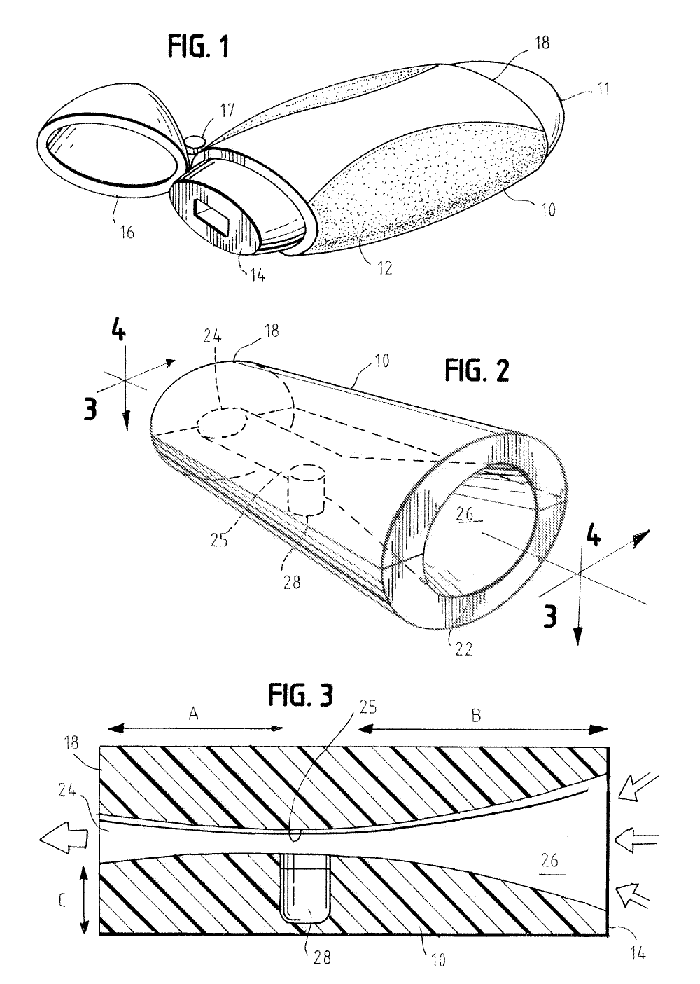

This disclosure takes advantage of flow energy of inspired air to disperse micronized particles packaged in a dosage form. In the air passageway of a dry powder inhaler, an air stream of air entering an inlet opening is constricted in a narrow portion, causing the velocity to increase. The increase in velocity induces a region of low pressure within the passageway. A reservoir, or charge of a dry powdered medicine is placed along the constriction. The flow energy of the air stream drives recirculating flow patterns adjacent and within the reservoir, or well, to fluidize the particles and draw the particles from the packaged dosage form and disperse the separated particles into the air stream. The passageway is then widened as it nears the exit of the passageway to reduce pressure drop within the inhaler. FIGS. 1-5 illustrate one embodiment of a dry powder inhalation device, generally designated as 8. Dry powder inhaler 8 includes a housing 10 including a gripping surface 12, an inlet end 14, and an outlet end 18. The inlet and outlet ends may include protective covers or caps 16, which may be secured by a hinge 17, or alternately have no hinge and be secured with a small interference fit or a snap fit.

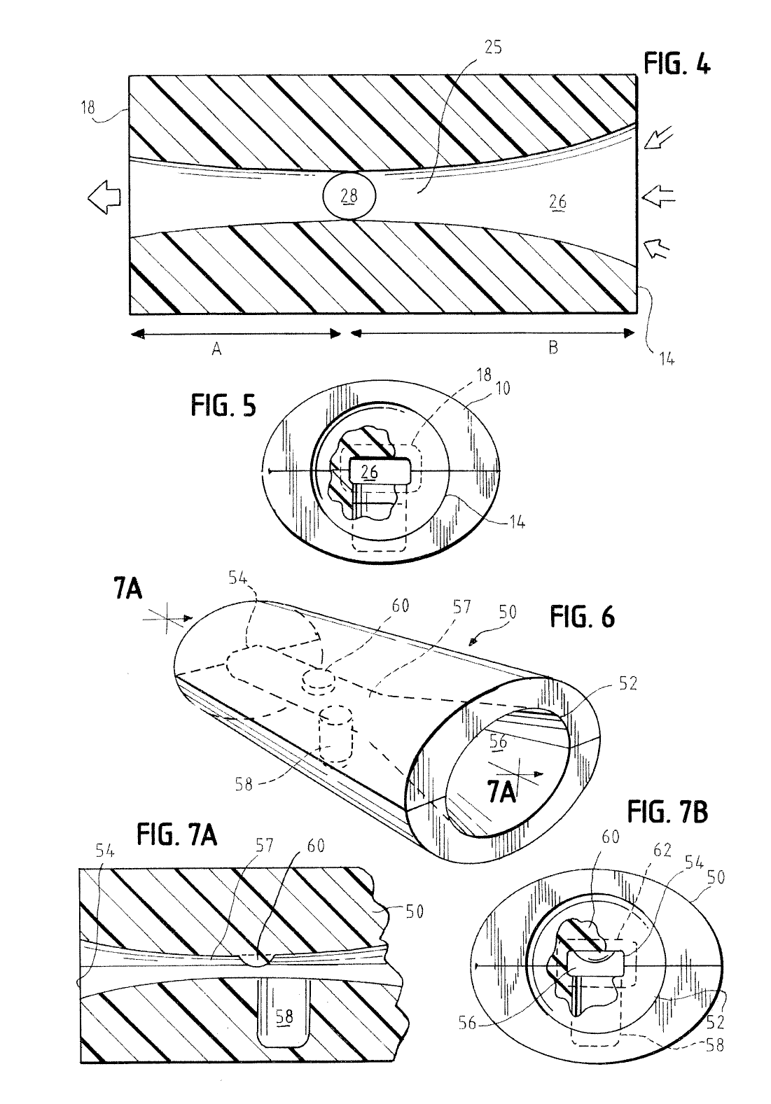

As seen in FIG. 2, housing 10 includes an air inlet end 14 with a large cross sectional area (FIG. 4), and an outlet end 18 with a smaller cross sectional area. The inhaler includes an internal air passageway 26 with a narrow portion 25, and a well 28 for a reservoir or supply of powder for the powder inhaler. In this embodiment, inlet end 14 cross sectional shape is circular or ovate as shown, while outlet end 18 cross sectional shape has the shape of a rounded rectangle. As also seen in FIG. 3, passageway 26 has a narrow portion near the middle, in the general vicinity of well 28. Air flow through the inhaler proceeds from the inlet end 14, through the narrow portion 25, and again out through the outlet 24 into the mouth and lungs of the person using the inhaler.

As seen in the top view of FIG. 4, in this embodiment, the width or diameter of well 28 approximates the width of the narrow portion 25 of the air passage. In the end view of FIG. 5, there is a clear view through the inhaler 10, i.e., a straight line from inlet end 14 to outlet end 18 through passageway 26. As seen in FIGS. 3-4, the distance from the inlet end 14 to the near end of well 28 is around 1.50, times the distance from the far side of well 28 to outlet end 18, thereby placing the well 28 closer to the outlet end 18 than the inlet end 14. In this embodiment, the air passageway is a smooth, tapering passage through the center of the housing.

The well 28 is preferably oval shaped. It has been found that orienting the major diameter in the direction of the airflow increases the efficiency of the release of particles from the well 28. In an embodiment, the well 28 is oval shaped and has a minor diameter of 3.0 mm and a major diameter of 4.0 mm. It has also been found that the ratio of the depth of the well to the length of the major diameter effects the efficiency of the dispersion. If the well 28 is too shallow or has a low depth to major diameter ratio, then the dose is swept from the well without the desired deagglomeration. In an embodiment the well has a depth of 5.0 mm and has a depth ratio greater than 0.5 and preferably is closer to 1.0.

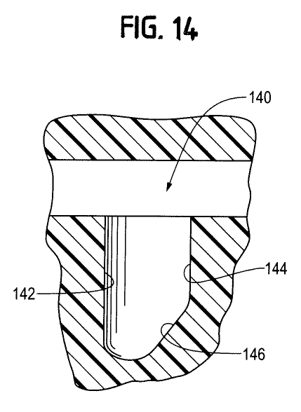

Referring to FIG. 14 in conjunction with FIG. 3, an embodiment of a well 140 has been shown to enhance the percentage of delivered dose. The well 140 includes a forward side which is upstream toward the inlet end 14 and is configured with a downwardly extending first section 144 forming a generally perpendicular orientation to the passageway 26. Below the first section 144 is an inclined section 146 which is angled in the downstream direction toward the outlet end 16. The downstream wall of the well is formed of one section 142 that is generally perpendicular to the passageway 26.

Referring back to FIGS. 1-3, the inlet end portion of the air passageway includes an opening formed in the inlet end 14 for receiving a stream of air into the air passageway 26. The inlet opening has a cross-sectional width that is greater than the cross-sectional width of the narrow, air flow restriction section 25, and thus tapers toward the narrow portion. In one embodiment the cross-sectional width of the inlet opening is about 8 to 12 mm in diameter. Although the cross-sectional shape of the inlet opening and the inlet end portion 14 are illustrated as being circular or ovate, the cross-sectional shape of the inlet opening and the inlet end portion also can be other simple closed curves. By way of example only the inlet end portion can also be formed as an ellipse with an aspect ratio of around 1:1 to 1:1.2.

The inlet end portion of the air passageway tapers or converges and transitions in shape in the direction of the narrow, air flow restriction section 25. The transition is defined by generally smooth walls and gradual shape changes to provide for smooth air flow from the inlet end into the general vicinity of the well 28. In the embodiment of FIGS. 1-5, the inlet end portion of the air passageway and the portion of the interior wall of body defining the passageway have a generally inwardly tapering shape converging toward the narrow portion 25 of the passage 26. The opening at the outlet end 18 also tapers toward in a mirror like fashion to the flow restriction section 25 in this embodiment, but the taper in the outlet section may be less pronounced,

As can best be appreciated in FIGS. 1-5, the cross sectional shape of the air passageway changes gradually from a generally circular cross section to a rounded rectangular as one moves from the inlet inward. Thus the taper of the passageway will vary depending on the orientation of the device but generally the taper from the inlet to the well portion is about 10 to 15 degrees. Moving from the outlet end 18 inward, the air passageway 26 changes gradually from an oval to the rounded rectangular with the taper ranging from 2 to 10 degrees.

The airflow restriction section 25 is located between the inlet end portion and the outlet end portion of the air passageway 26. The airflow restriction section 25 has a cross-sectional width that is less than the cross-sectional width of the inlet end portion 22 and can be less than the cross-sectional width of the outlet end portion 24. As noted above in the illustrated embodiment and for example only, the cross-sectional shape of the flow restriction section is a generally rectangular shape having rounded corners. In the vicinity of the well 28 the cross sectional height is approximately 1.86 mm and a cross sectional width of about 5 mm. The cross-sectional shape of the flow restriction section can also be other planar closed simple curves

Although the flow restrictor section 25 can be other shapes the rounded rectangular is preferred. It is generally believed that the velocity of an air stream through a passageway is highest at the point midway between the surfaces of the passageway. Therefore having a rounded rectangular shape with the shorter sides extending in the same direction as the extension of the well 28 acts to place this mid-point and higher velocity closer to the opening of the well 28 than many other shapes.

Again by way of example, the cross sectional area of the opening defined by the inlet 14 may vary from about 0.075 square inches to about 0.085 square inches, and the cross sectional area defined by the outlet 18 varies from about 0.022 square inches to about 0.032 square inches. The cross section of the narrow portion 25 varies from about 0.011 square inches to about 0.020 square inches.

The airflow restriction section 25 acts as a choke or restrictor on the flow of air. As a stream of air flows from the inlet portion 22 into the narrow passageway portion 25, the velocity increases, as the same mass of air is forced to flow into the passageway with a smaller cross section. When this faster air passes the well 28, holding a reservoir or charge of powdered inhalant, the flow energy drives a generally looping recirculating air stream pattern in the well 28. The air stream separates and fluidizes the particles and draws the particles from the well and disperses those particles into the air stream. The particles then flow through the outlet 18 into the mouth and lungs of the person using the inhaler. In the embodiments disclosed herein, there are few restrictions downstream of the well 28 or reservoir to impede movement of the air and the powder. Also surfaces on which the particles may impinge are minimized.

FIGS. 6 and 7A-7B disclose a second embodiment. As seen in the perspective view of FIG. 6, the inhaler housing 50 includes an inlet section 52, an outlet section 54 and a passageway 56 through the housing. Unitary passageway 56 includes a narrow portion 57 in the middle and outlet sections. There is a well 58 for a reservoir of powder, and a downwardly depending flow restriction 60 in the general vicinity of the well 58 and in an embodiment it is placed slightly downstream of the mid portion of the well 58. As also seen in the side view of FIG. 7A, in an illustrated embodiment at least a portion of well 58 and restriction 60 overlap. That is, a portion of restrictor 60 is above a portion of well 58, which are respectively, on the top and bottom of passageway 56, and on opposite sides of the passageway.

Having the downwardly depending restriction over the well 58 causes the air stream over a portion of the well 58 to further increase in velocity as it flows over the well 58. This acts to increase the re-circulating air patterns formed in the well to fluidize the particles and draw the particles from the packaged dosage form and disperse the separated particles into the air stream.

In prior art, there are also one or more impact plates. The impact plates are typically intended to de-agglomerate particles that are sticky and have a tendency to clump or aggregate together and also separate the active pharmaceutical ingredient from the lactose carriers. While impact plates help to solve this problem, but they also present additional surfaces onto which the sticky particles may adhere, thus reducing the efficiency of the inhaler. Impact plates may also act to impede the flow of air thereby contributing to the pressure drop within the inhaler.

The inhaler 8 may be formed from many different materials. In a preferred embodiment, the housing 10 is formed of a polypropylene. To assist in preventing the clinging of the particles to the housing 10 an antistatic additive may also be used. One particular antistatic additive is ENTIRA from Dupont. In an embodiment the antistatic additive may be added in a 10%-30% concentration and in a preferred embodiment in about a 20% concentration. Other materials may include polycarbonate, polystyrene, nylon, ABS, high density polyethylene (HDPE), acetal, PBT, PETG, various thermoplastic elastomers, and/or combinations thereof both with and without antistatic additives.

In the graphs that are discussed below, embodiment 1 is the embodiment of FIGS. 1-5, while embodiment 2 is the embodiment of FIGS. 6 and 7A-7B.

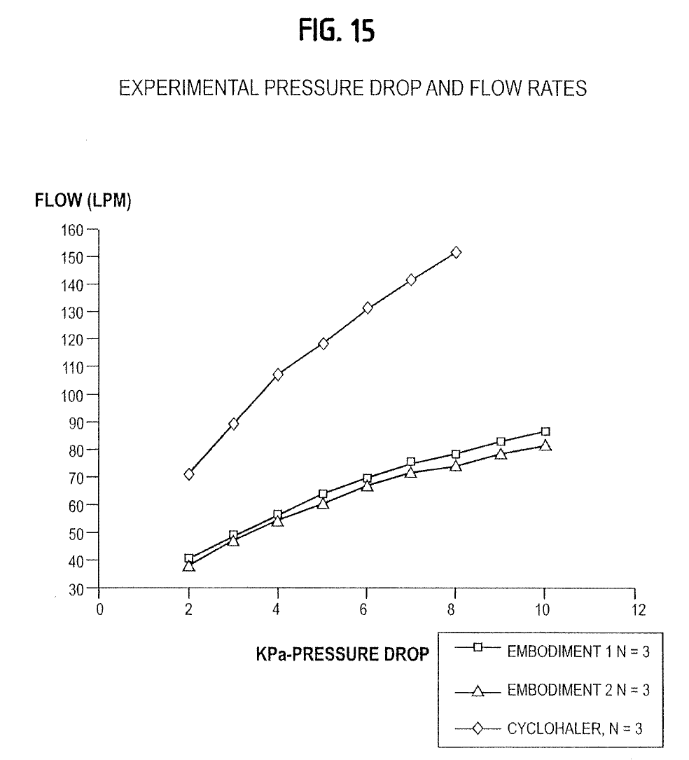

Referring to FIG. 15 a graph illustrates the variation in flow rate, in liters per minute (LPM) generated through the inhaler with the range of inspiration efforts which are typically applied by patients for the two embodiments and a commercially available CYCLOHALER. The graph demonstrates relatively small change in flow rate (40-65 LPM) with the variation in relevant patient clinical inspiration efforts (2-6 kPa) which are applied to embodiment 1 and embodiment 2. In contrast for the CYCLOHALER there is a much larger variation in flow rate (70-130 LPM) for the same range inspiration effort. The small variation in flow rate through the embodiments 1 and 2 under the different inspiratory efforts typically employed by patients improves the consistency in the delivery of the therapeutic agent not only to a single patient who may vary the inspiration effort but also between patients having unique inspiration efforts. This test characterizes the new inhaler designs advantages in more effectively controlling the delivery of the therapeutic agent while minimizing effects due to patient-to-patient variability during inhalation.

Another important aspect of performance of the inhaler is the ability to transport a dose from the inhaler to the lungs of the patient. This overall performance depends on the interaction of a number of circumstances. However, it is acknowledged that good performance generally requires that the powder should have few impacts as it traverses from the inhaler to the person, and that the powder should not agglomerate during this traverse.

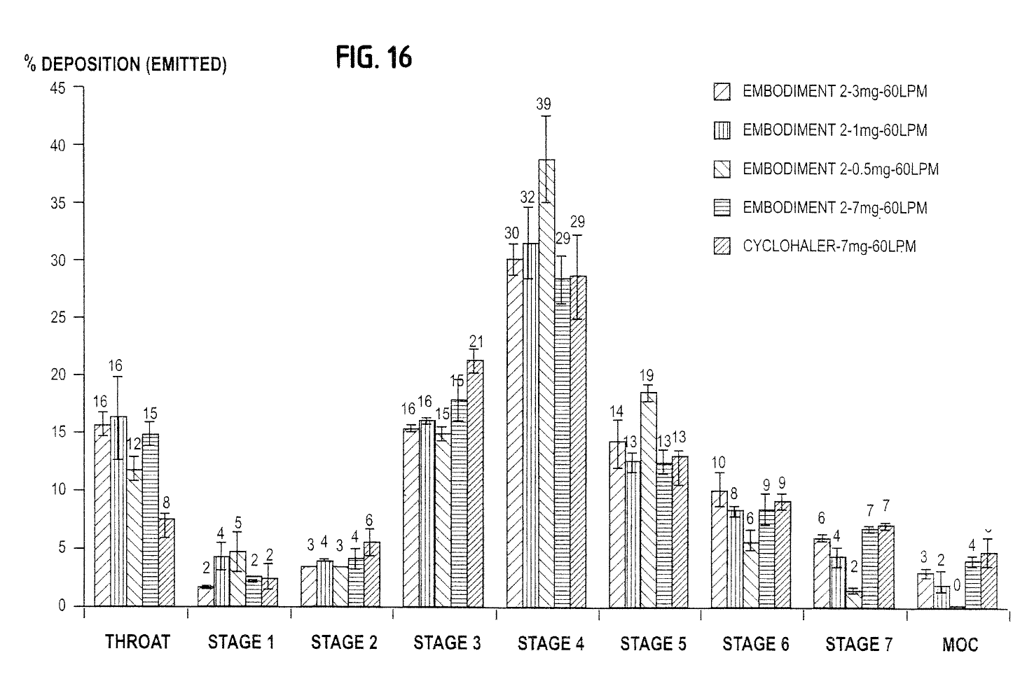

FIG. 16 depicts tests results from inhaler tests using embodiments 1 and 2 as discussed above, and the CYCLOHALER. These test results were obtained using a a Next Generation Pharmaceutical Impactor Model 170 (NGI), which is available from MSP Corporation Inc., Minneapolis, Minn. USA. As seen in FIG. 16 the delivery profile embodiments 1 and 2 outperform the CYCLOHALER inhaler in terms of the dose ranges that the embodiments deliver (0.5-7 mg). The dry powder being dispersed is composed of small spherical particles of insulin with a size range of 1.0-6.0 μm. Moreover, the delivery by embodiments 1 and 2 occur with no actuation failures that are associated with capsule or mechanical dispensing DPIs and maintains consistence aerodynamic performance.

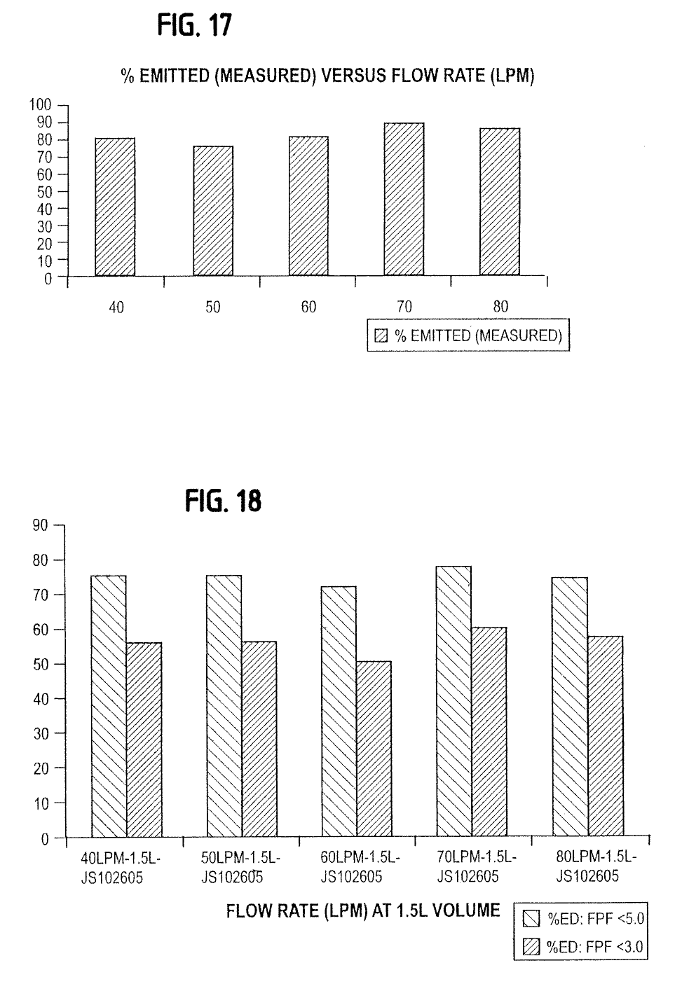

As is shown in FIG. 17 the embodiments 1 and 2 disperses the particles as effectively as the CYCLOHALER. However, embodiments 1 and 2 have the added benefit of flexible dosing with consistent performance. This testing shows the superiority of the new inhaler designs.

Another important aspect of performance of the inhaler is the amount of the dose which is emitted from the inhaler. Many therapeutic agents are very expensive and any dose amount that remains in an inhaler increases the cost to the patient of administering a desired dose.

Tests were conducted to determine the percentage of emitted dose using embodiments 1 as discussed above, and the CYCLOHALER. These tests were conducted at low dose levels which pronounced the effects of the inhaler on the emitted dose as one would assume that the inhaler would retain generally the same amount of dose regardless of the size of the dose and so the smaller the dose the more this retained amount will show up in a larger reduction in the percentage of emitted dose.

The CYCLOHALER inhaler was evaluated for drug delivery performance using PROMAXXÒ Recombinant Human Insulin Inhalation Powder (RHIIP) at a 2 mg nominal load. The DPI was tested at 60 liters per minute with an eight stage non-viable Andersen Cascade Impactor (ACI). Analysis was performed by High Pressure Liquid Chromatography.

TABLE 1
RESULTS

| | | % | % of Load | % of Load |
|---|---|---|---|---|
| Test | Load | Emitted | Remaining in | Remaining in |
| Reference | (mg) | Dose | Capsule | Device |
| PBS3 WK3 | 1.866 | 59 | 25.3 | 15.4 |
| PBS7 WK2 | 1.887 | 70 | 19.3 | 10.6 |
| PBS3 WK5 | 1.864 | 54 | 28.6 | 17.1 |
| PBS7 WK5 | 1.855 | 61 | 31.3 | 8.0 |
| PBS7 WK3 | 1.846 | 66 | 22.5 | 11.8 |
| PBS7 WK1 | 1.819 | 62 | 26.4 | 11.4 |

Results indicate the CYCLOHALER Dry Powder Inhaler delivers a low emitted dose with a high degree of variability and retains a high percentage of PROMAXX RHIIP in the capsule of the nominal 2 mg load at 60 liters per minute.

Embodiment 1 of the present inhaler was also evaluated for drug delivery performance using PROMAXX Recombinant Human Insulin Inhalation Powder (RHIIP) at a 1 mg nominal load. The DPI was tested at 52 liters per minute. These test results were obtained using a a Next Generation Pharmaceutical Impactor Model 170 (NGI), which is available from MSP Corporation Inc., Minneapolis, Minn.

TABLE 2
RESULTS

| | | | % of Load | % of Load |
|---|---|---|---|---|
| | | % Emitted | Remaining | Remaining in |
| Lot Number | Load (mg) | Dose | inWell | Device |
| 019407-B | 1 | 79.3 | 15.7 | 1.7 |
| 100407-B | 1 | 82.2 | 15.5 | 2.0 |
| 101807-B | 1 | 80.8 | 15.4 | 2.3 |
| Average | 1 | 80.7 | 15.6 | 2.0 |
| Std. Dev | | 1.4 | 0.2 | |

Results indicate the Embodiment 1 of the present inhaler delivers a much higher emitted dose with a low degree of variability and retains a low percentage of PROMAXX RHIIP in the well using the nominal 1 mg load at 52 liters per minute.

FIG. 16 depicts tests results from inhaler tests using embodiments 1 and 2 as discussed above, and the CYCLOHALER. These test results were obtained using a a Next Generation Pharmaceutical Impactor Model 170 (NGI), which is available from MSP Corporation Inc., Minneapolis, Minn. USA As seen in FIG. 16 the delivery profile embodiments 1 and 2 outperform the CYCLOHALER inhaler in terms of the dose ranges that the embodiments deliver (0.5-7 mg). The dry powder being dispersed is composed of small spherical particles of insulin with a size range of 1.0-6.0 μm. Moreover, the delivery by embodiments 1 and 2 occur with no actuation failures that are associated with capsule or mechanical dispensing DPIs and maintains consistence aerodynamic performance.

As is shown in FIG. 17 the embodiments 1 and 2 disperses the particles as effectively as the CYCLOHALER. However, embodiments 1 and 2 have the added benefit of flexible dosing with consistent performance. This testing shows the superiority of the new inhaler designs.

Many DPI have a dose capacity/dependency in which the DPI reduces its ability to de-agglomerate the powder at higher dose levels as well as loose its ability for the dose to clear the device at clinical relevant inspiratory flow rates and inhalation volumes. Further testing was also accomplished with embodiment 2. As shown in FIGS. 17-18 testing demonstrated the ability of embodiment 2 to delivery the spherical particles at high dose levels without affecting the respirable dose fractions under clinical relevant flow rates and low inhalation volume. In a first series of in vitro tests, a dose of about 7 mg was placed into the reservoir, and actuation was tested using the NGI. A 1.5 liter (1.5 L) “breath” was used at peak inspiration flows (PIF) from 40 liters per minute (LPM) to 80 LPM. Many adult males have an inspiration capacity from 2.2 to 4 liters, while adult females may range from 1.5 to 3 liters. The testing here used this lowest capacity, 1.5 liters, and is thus conservative.

The result of this first series is depicted in FIG. 17 As FIG. 17 shows, there is a slight upward trend as the PIF goes from 40 LPM to 80 LPM, with the highest percent emitted at 70 LPM. This graph demonstrates the effectiveness of the new inhaler in releasing high doses of powdered medicine from the reservoir to the patient at both low and high inspiration rates. 80% of the dose was emitted at the median rate of 60 LPM. In other tests, the effectiveness of the new design was shown. Moreover the embodiments significantly reduce the correlation between delivered dose and patient specific inhalation patterns.

Generally lower flow rates provide less dispersion energies for de-agglomerating the powder which adversely impacts the dose delivery that is respirable. While FIG. 17 depicts the overall release of medicine, FIG. 18 demonstrates how well the medicine is dispersed into desired particle sizes at various clinical relevant flow rates. FIG. 18 shows the mass fractions of the particles by size range that are dispersed at two different flow rates by the inhaler of the present embodiment. To the extent that agglomeration occurs we would expect the fraction that is respirable (<5 um and <3 um (for deep lung delivery) to decrease with a reduction in flow rate given less dispersion energy. However, in this series of tests, this did not occur, showing the superiority of the new design.

The data shows results from inspiration of 1.5 liters at flows from 40 LPM to 80 LPM. Across all rates, about 75% of the emitted dose had a fine particle fraction of less than 5 μm, with a range from 71 to 76%. The fraction of fines below 3.0 μm was also impressive, ranging from 49 to 58%. In the median rate, 60 LPM, about 70% of the emitted dose had a particle size of less than 5 μm, and about 50% at less than 3 μm. In summary, the capability of the inhaler to disperse particles which have a tendency to agglomerate has been found to be very weakly dependent on the flowrate (input energy) for the range of flowrates investigated. This weak dependency points to robustness of the design as a delivery and dispersion or de-agglomeration engine.

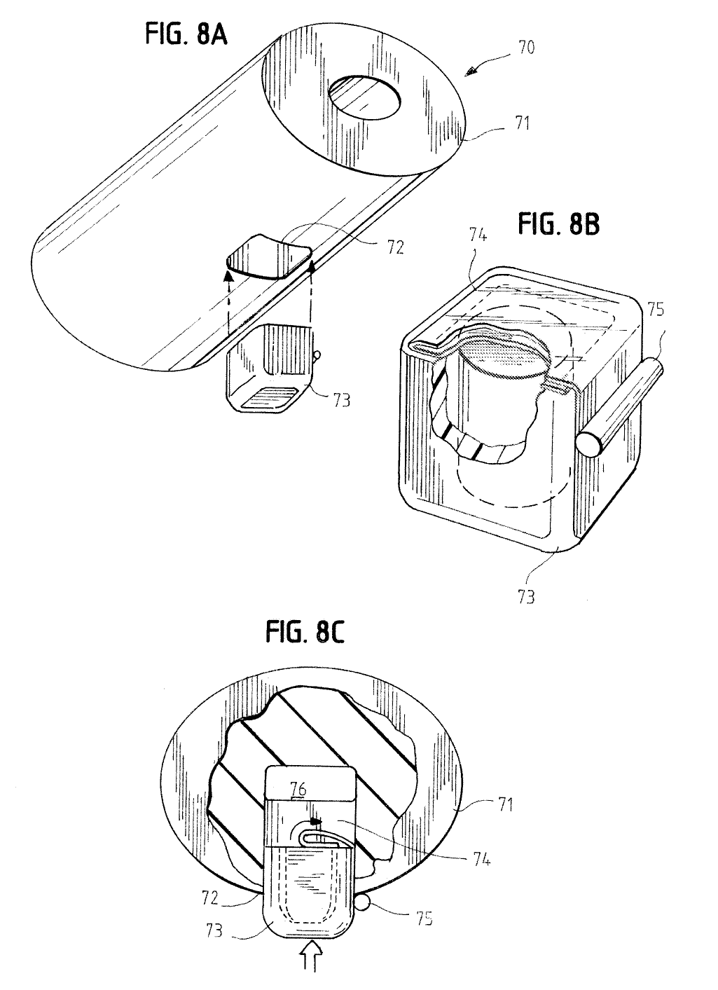

There are many other embodiments of the improved inhaler which may employ different designs for placement of a package having a desired does. FIGS. 8A-8C depict an inhaler 70 in which insertion of a inhaler dose package 73 also opens the package and prepares it for inhaling by the user. In this embodiment, inhaler 70 includes an aperture 72 for insertion of a powder package 73. Powder package 73, which includes a measured dose of a dry powder, such as insulin, includes a seal 74 on the top of the package, and also includes an opening lip 75. When the package is inserted into the inhaler, the inhaler fixes to catch 75, and holds it in place such that seal 74 is removed by relative motion between 70 & 73, as shown by the arrow in FIG. 8C, thus opening the package. Aperture 72 may include a stop (not shown) for controlling the depth of insertion into the inhaler. When the user is ready, a breath is drawn and the powder is pulled or vacuumed through the passageway 76 and breathed into lungs of the user for either fluid path embodiment 1 or 2.

The embodiments of FIGS. 1-7, and also the embodiment of FIGS. 8A and 8C, include a smooth, unitary passageway, broken only by the well or reservoir and the restriction, if used. These embodiments have a virtually no other interruptions or breaks in their surfaces to catch air, introduce drag, or otherwise interfere with a smooth and uninterrupted flow of air. It is not necessary that only smooth, unitary air passages be used in the improved inhalers. As shown in the embodiments discussed below, the housings which include the air passages may be made in two or more parts. When the parts are joined, there are inevitably at least minute gaps or overlaps between the parts, such as between top and bottom halves, or between a main housing and one or more inserts used to capture the medicament packet or dose. These gaps and mismatches should obviously be kept to a minimum to limit unpredictable disruption to air flow. The purpose of inhalers is to deliver a predictable flow every time from every inhaler. If the discontinuities are not predictable, and vary from device to device, then the devices will not have uniform, predictable performance. Accordingly, discontinuities and gaps should be kept to a minimum, and seals on the powder packages or cartridges should be effective to close any gaps where they are used.

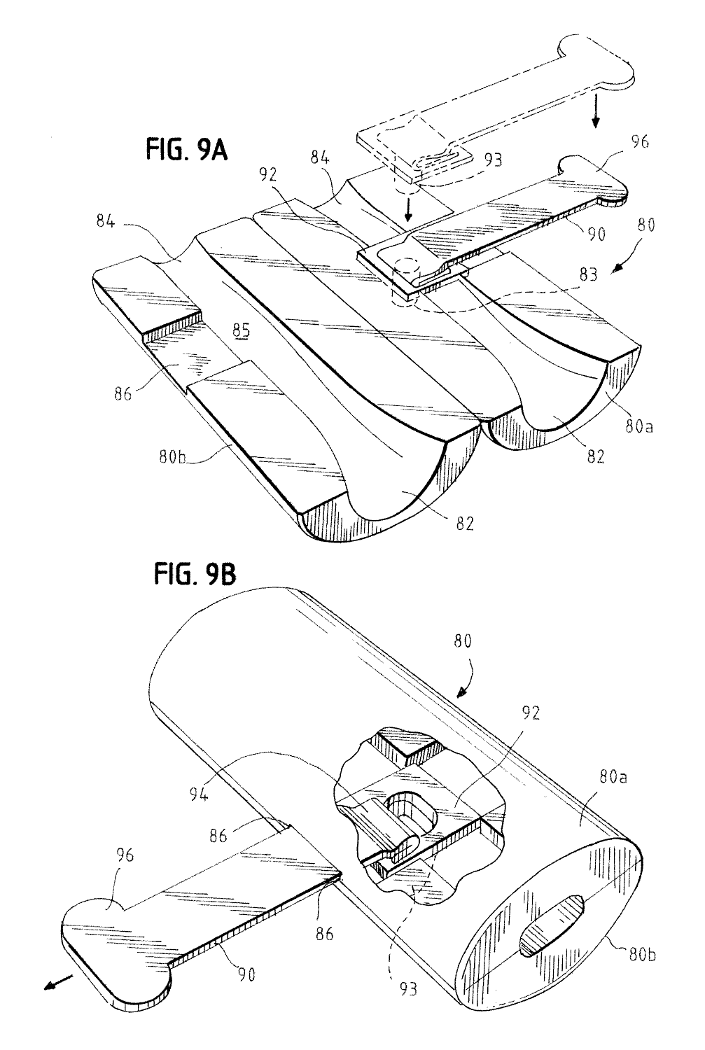

In another embodiment, the housing has two halves, which are assembled for use. FIGS. 9A-9B depict an inhaler 80, with a housing that includes a lower half 80a and an upper half 80b. The lower half 80a also includes a well 83 for the dose. Upper half 80b closely matches lower half 80a, except for the well. Both halves include an inlet portion 82, an outlet portion 84, and a narrow portion 85. Both halves also include a relief 86 and a lip seal 88 for sealing the dose package used. The fluid path in the inhaler may be either fluid path embodiment, 1 or 2, previously depicted, with an inlet portion having a greater diameter or cross section than the outlet portion, and with the narrow portion having a cross section or diameter that is smaller than the other portions of the passageway.

The dose package 90 includes a distal portion 92, a reservoir 93, a seal 94, and a handle and opener 96. When a user wishes to inhale a medicament, the user opens the inhaler 80 and places reservoir 93 into well 83, and closes the inhaler. The user than pulls on opener 96. The opener then draws the seal 94 from the top of the reservoir. The seal 94 may be designed to seal the relief area 86 when the handle and opener 96 is completely withdrawn for the inhaler, for easier use. This design allows insertion of the reservoir without touching the reservoir or the well by the user or by a caregiver, and thus completely avoids any contamination that could result from touching an upper surface of the medicament package.

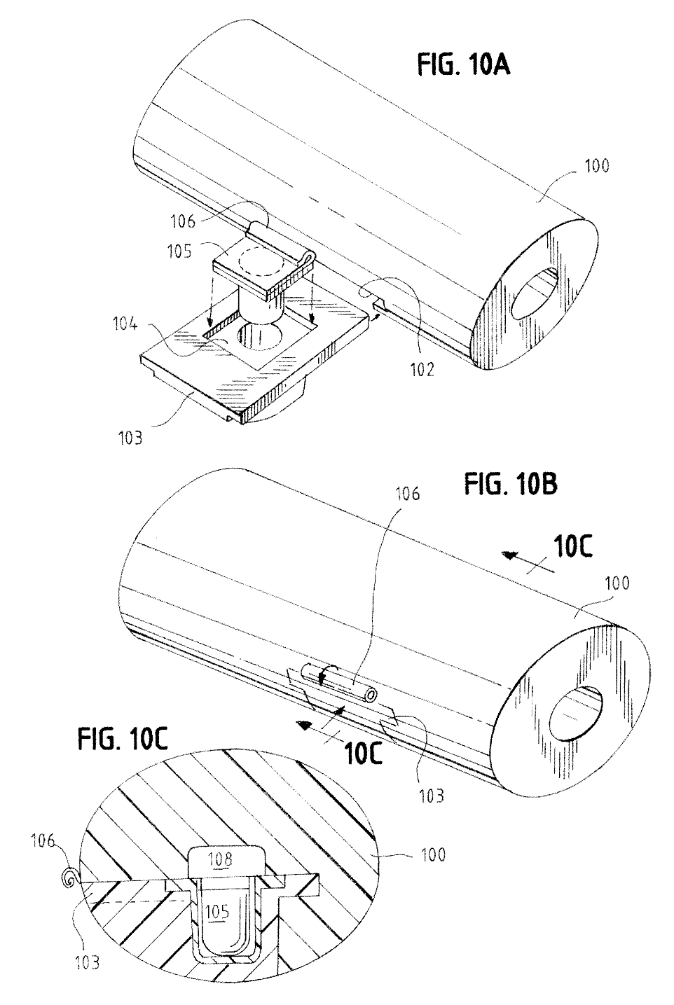

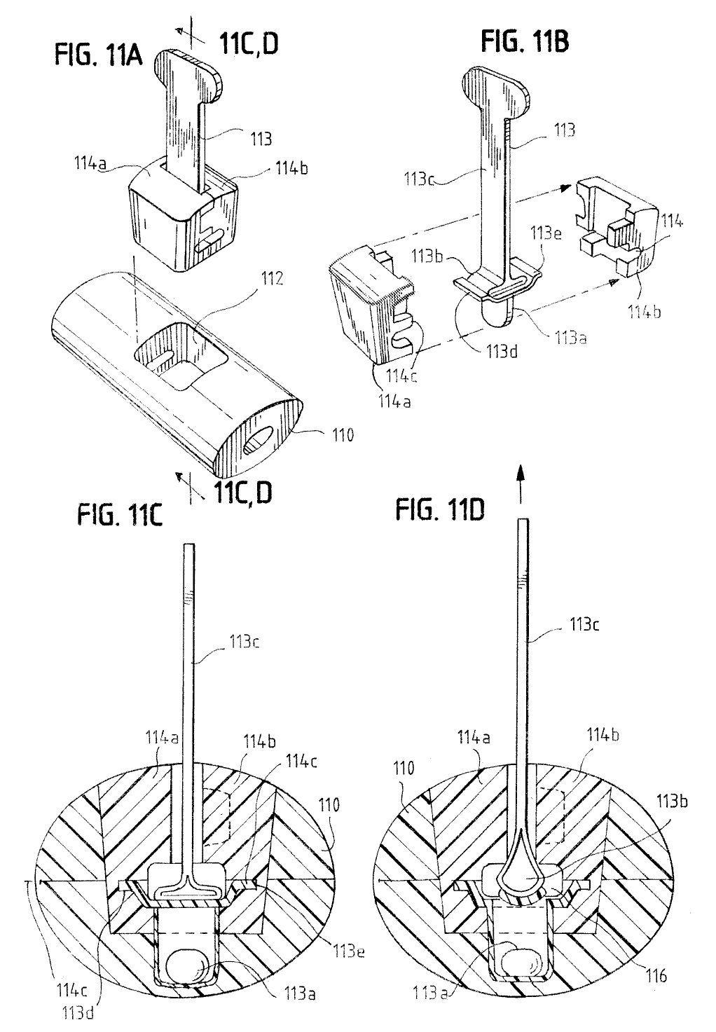

Other embodiments may also use no-touch reservoirs, such as that shown in FIGS. 11A-11D. In this embodiment, inhaler 110 includes an aperture 112 for insertion of a medicament package 113 with its housing 114a, 114b. While the embodiment of FIGS. 10A-10B uses a medicament pouch with a side-pull seal, the medicament 113 in this embodiment uses a straight-pull seal, straight in the direction of the axis of the package. As seen in FIGS. 11A-11B, package 113 includes a reservoir 113a of a medicament, and also includes a top seal 113b, along with a combination handle and opener 113c. Package 113 is placed between housing halves 114a, 114b, and lips 113d, 113e of package 113 are retained by sides 114c of the housings. The assembly is then inserted into aperture 112 of the inhaler 110.

After assembly, the package is opened as depicted in FIGS. 11C-11D. Housing halves 114a, 114b are held within housing 110 by the user, or they may be held by a reversible snap fit. The user pulls on combination handle/opener 113 while holding housing 114a, 114b within the inhaler. Seal 113b opens as the opener 113 is withdrawn, exposing the reservoir 113a to the force of inhaled air in passageway 116 of the inhaler when the user breathes in the dose. This embodiment also features minimal touching by the user, since only the handle or opener need be touched, along with housing halves 114a, 114b, and the inhaler 110 itself.

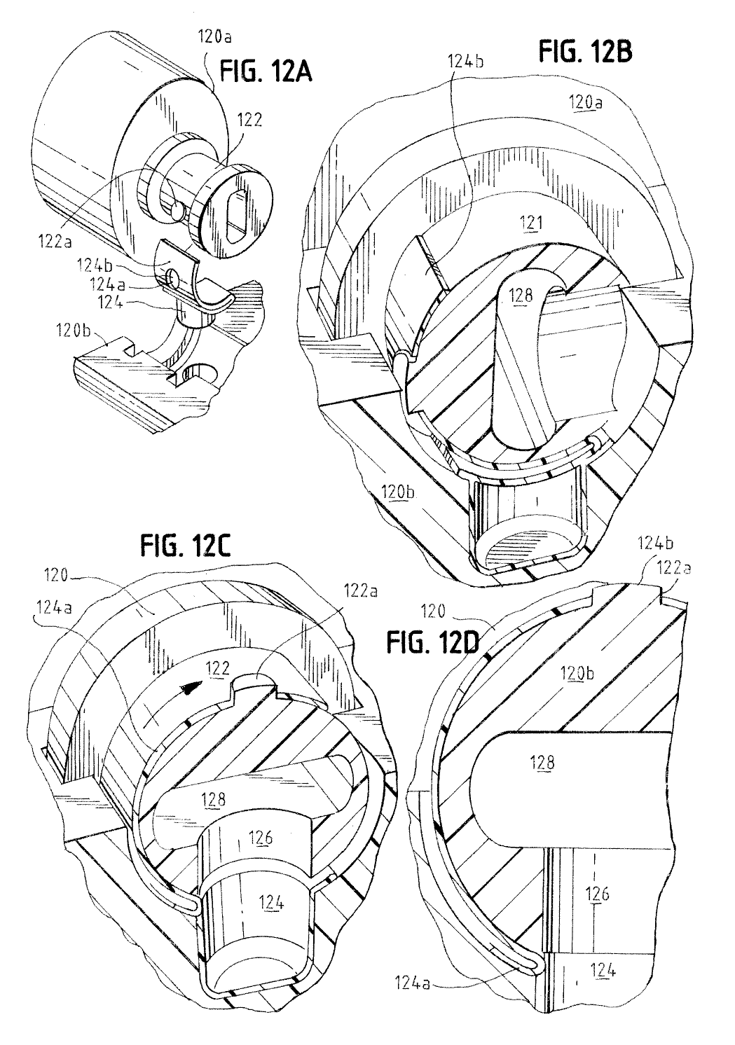

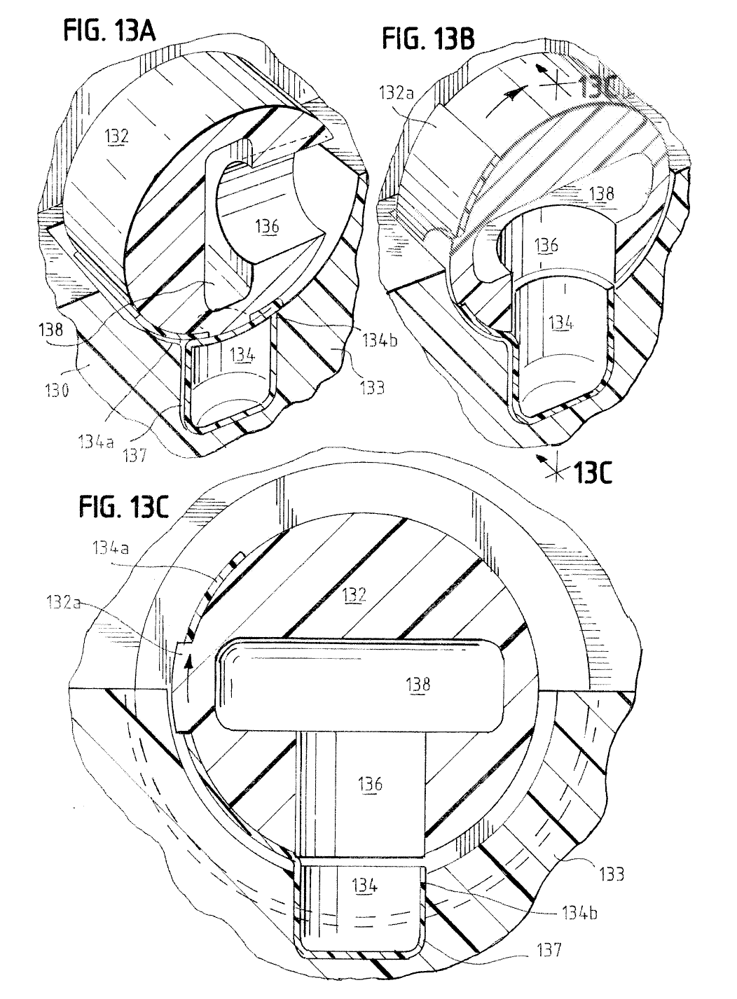

The embodiments of FIGS. 10A-10B and 11A-11D used straight sideways or top-side insertion of the medicament dose or pouch. Other embodiments use a rotary motion for opening of the pouch once it is assembled to the inhaler, as shown in FIGS. 12A-12D and in FIGS. 13a-13D.

In FIG. 12A, the inhaler housing has two portions, a proximal housing 120a, which includes the outlet end (not shown) and a distal housing 120b, which includes the inlet end 120b. Outlet end 120a is machined or preferably, molded with several diameters on one end, as shown. Central portion 122 of proximal housing 120a includes a round boss 122a and an interface portion 121 with a larger diameter, the central portion configured to receive a medication package 124 and the central portion also configured for mating with distal housing 120b. Boss 122a may be considered an end portion of a “rotating rod” for pulling the seal away from the pouch.

In use, the medication package 124, with seal 124a and aperture 124b is placed on central portion 122 and boss 122a is placed through aperture 124b, as shown in FIG. 12B. Distal housing 120b is than assembled to proximal housing 120a, which is rotated clockwise, as shown in FIG. 12C. Rotation of the proximal housing removes seal 124a while the package 124 itself is held rigidly within well 127 in distal housing 120b. The seal may be of any desired length to fit with a desired rotation, but 90° is a convenient rotation, and as shown in FIG. 12D, at the completion of a 90° rotation, the seal 124a has been removed from the package, and the inhaler is ready for use.

In another embodiment, depicted in FIGS. 13A-C, the housing geometry is simplified. Inhaler 130 includes proximal half 132 and distal half 133. Proximal half 132 includes a surface which is bonded with tab 134a of medicament pouch 134. The proximal half 132 also includes air passageway 138 and an upper portion 136 of a well for the pouch. Distal half 133 is split, including bottom half 133a that includes lower portion 137 of the well. Upper half 133b and lower half 133a both include portions of an air inlet 131. The pouch 134 is placed on proximal half 132 and the distal half 133 is then assembled by a clamping action to the proximal half.

As noted, the medicament pouch includes a reservoir and a tab 134a. The top of the pouch, 134, also seals against the opening, 136, of proximal end 132. Pouch 134 is cylindrical in this embodiment, but may also be elliptical. There are many embodiments of the inhaler, of which this description provides only a few.

Although the dry powder utilized in the tests discussed above was comprised of insulin, other pharmaceutical substances or other therapeutic agents could also be utilized in the inhaler. The therapeutic agent can be a biologic, which includes but is not limited to proteins, polypeptides, carbohydrates, polynucleotides, and nucleic acids. The protein can be an antibody, which can be polyclonal or monoclonal. The therapeutic can be a low molecular weight molecule. In addition, the therapeutic agents can be selected from a variety of known pharmaceuticals such as, but are not limited to: analgesics, anesthetics, analeptics, adrenergic agents, adrenergic blocking agents, adrenolytics, adrenocorticoids, adrenomimetics, anticholinergic agents, anticholinesterases, anticonvulsants, alkylating agents, alkaloids, allosteric inhibitors, anabolic steroids, anorexiants, antacids, antidiarrheals, antidotes, antifolics, antipyretics, antirheumatic agents, psychotherapeutic agents, neural blocking agents, anti-inflammatory agents, antihelmintics, anti-arrhythmic agents, antibiotics, anticoagulants, antidepressants, antidiabetic agents, antiepileptics, antifungals, antihistamines, antihypertensive agents, antimuscarinic agents, antimycobacterial agents, antimalarials, antiseptics, antineoplastic agents, antiprotozoal agents, immunosuppressants, immunostimulants, antithyroid agents, antiviral agents, anxiolytic sedatives, bone and skeleton agents, astringents, beta-adrenoceptor blocking agents, cardiovascular agents, chemotherapy agents, corticosteroids, cough suppressants, diagnostic agents, diagnostic imaging agents, diuretics, dopaminergics, enzymes and enzyme cofactors, gastrointestinal agents, growth factors, hematopoietic or thrombopoietic factors, hemostatics, hematological agents, hemoglobin modifiers, hormones, hypnotics, immunological agents, antihyperlipidemic and other lipid regulating agents, muscarinics, muscle relaxants, parasympathomimetics, parathyroid hormone, calcitonin, prostaglandins, radio-pharmaceuticals, sedatives, sex hormones, anti-allergic agents, stimulants, steroids, sympathomimetics, thyroid agents, therapeutic factors acting on bone and skeleton, vasodilators, vaccines, vitamins, and xanthines. Antineoplastic, or anticancer agents, include but are not limited to paclitaxel and derivative compounds, and other antineoplastics selected from the group consisting of alkaloids, antimetabolites, enzyme inhibitors, alkylating agents and antibiotics.

Exemplary proteins, include therapeutic proteins or peptides, or carrier proteins or pep-tides, including GCSF; GMCSF; LHRH; VEGF; hGH; lysozyme; alpha-lactoglobulin; basic fibroblast growth factor basic fibroblast growth factor; (bFGF); asparaginase; tPA; urokin-VEGF; chymotrypsin; trypsin; ase; streptokinase; interferon; carbonic anhydrase; ovalbumin; glucagon; ACTH; oxytocin; phosphorylase b; alkaline phos-secretin; vasopressin; levothyroxin; phatase; beta-galactosidase; parathyroid hormone, calcitonin; fibrinogen; polyaminoacids (e.g., DNAse, alpha1 antitrypsin; polylysine, polyarginine); angiogenesis inhibitors or pro-immunoglobulins (e.g., antibodies); moters; somatostatin and analogs; casein; collagen; soy protein; and cytokines (e.g., interferon, gelatin. interleukin); immunoglobulins.

Exemplary hormones and hormone modulators include insulin, proinsulin, C-peptide of insulin, a mixture of insulin and C-peptide of insulin, hybrid insulin cocrystals, growth hormone, parathyroid hormone, luteinizing hormone-releasing hormone (LH-RH), adrenocorticotropic hormone (ACTH), amylin, oxytocin, luteinizing hormone, (D-Tryp6)-LHRH, nafarelin acetate, leuprolide acetate, follicle stimulating hormone, glucagon, prostaglandins, steroids, estradiols, dexamethazone, testosterone, and other factors acting on the genital organs and their derivatives, analogs and congeners.

Exemplary hematopoietic or thrombopoietic factors include, among others, erythropoietin, granulocyte colony stimulating factor (G-CSF), granulocyte-macrophage stimulating factor (GM-CSF) and macrophage colony stimulating factor (M-CSF), leukocyte proliferation factor preparation, thrombopoietin, platelet proliferation stimulating factor, megakaryocyte proliferation (stimulating) factor, and factor VIII.

Exemplary therapeutic factors acting on bone and skeleton and agents for treating osteoporosis include calcium, alendronate, bone GLa peptide, parathyroid hormone and its active fragments, histone H4-related bone formation and proliferation peptide and their muteins, derivatives and analogs thereof.

Exemplary enzymes and enzyme cofactors include: pancrease, L-asparaginase, hyaluronidase, chymotrypsin, trypsin, tPA, streptokinase, urokinase, pancreatin, collagenase, trypsinogen, chymotrypsinogen, plasminogen, streptokinase, adenyl cyclase, and superoxide dismutase (SOD).

Exemplary vaccines include Hepatitis B, MMR (measles, mumps, and rubella), and Polio vaccines.

Exemplary growth factors include nerve growth factors (NGF, NGF-2/NT-3), epidermal growth factor (EGF), fibroblast growth factor (FGF), insulin-like growth factor (IGF), transforming growth factor (TGF), platelet-derived cell growth factor (PDGF), hepatocyte growth factor (HGF) and so on.

Exemplary agents acting on the cardiovascular system include factors which control blood pressure, arteriosclerosis, etc., such as endothelins, endothelin inhibitors, endothelin antagonists, endothelin producing enzyme inhibitors vasopressin, renin, angiotensin I, angiotensin II, angiotensin III, angiotensin I inhibitor, angiotensin II receptor antagonist, atrial naturiuretic peptide (ANP), antiarrythmic peptide and so on.

Exemplary factors acting on the central and peripheral nervous systems include opioid peptides (e.g. enkephalins, endorphins), neurotropic factor (NTF), calcitonin gene-related peptide (CGRP), thyroid hormone releasing hormone (TRH), salts and derivatives of TRH, neurotensin and so on.

Exemplary factors acting on the gastrointestinal system include secretin and gastrin.

Exemplary chemotherapeutic agents, such as paclitaxel, mytomycin C, BCNU, and doxorubicin.

Exemplary agents acting on the respiratory system include factors associated with asthmatic responses, e.g., albuterol, fluticazone, ipratropium bromide, beclamethasone, and other beta-agonists and steroids.

Exemplary steroids include but are not limited to beclomethasone (including beclomethasone dipropionate), fluticasone (including fluticasone propionate), budesonide, estradiol, fludrocortisone, flucinonide, triamcinolone (including triamcinolone acetonide), and flunisolide. Exemplary beta-agonists include but are not limited to salmeterol xinafoate, formoterol fumarate, levo-albuterol, bambuterol, and tulobuterol.

Exemplary anti-fungal agents include but are not limited to itraconazole, fluconazole, and amphotericin B.

Numerous combinations of active agents may be desired including, for example, a combination of a steroid and a beta-agonist, e.g., fluticasone propionate and salmeterol, budesonide and formoterol, etc

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. An inhalation device, comprising:
a tapering air passageway having an inlet end, a narrow portion, and an outlet end, wherein an inlet cross section of the inlet end is larger than an outlet cross section of the outlet end, and a cross section of the narrow portion is smaller than the inlet and outlet cross sections;
an air flow restriction between the inlet end and the outlet end, the air flow restriction placed at least partly in the narrow portion of the air passageway; and
a well having an opening disposed along the air passageway at the narrow portion and configured to receive a dose of an inhalable powder, the well configured so that a flow of inspired air through the air passageway draws the inhalable powder out of the well and through the inhalation device.

2. The inhalation device of claim 1, wherein at least a portion of the air passageway comprises a straight line between the inlet end and the outlet end.

3. The inhalation device of claim 1 wherein the inlet cross section is at least twice the outlet cross section.

4. The inhalation device of claim 1 wherein a distance from the inlet end to the inlet side of the air flow restriction is at least 1.5 times a distance from an opposite end of the air flow restriction to the outlet end.

5. The inhalation device of claim 1 wherein the restriction extends at least halfway between opposite sides of the narrow portion.

6. The inhalation device of claim 1 wherein a center of the restriction is downstream of a center of the well or at about ½ to ¾ of a distance between the inlet end and outlet ends.

7. The inhalation device of claim 1, wherein the restriction has a shape of a half of a football and is oriented with a long axis along the direction of air flow.

8. The inhalation device of claim 1, wherein a widest portion of the restriction occupies about 30% of the cross section of the narrow portion.

9. The inhalation device of claim 1, wherein a vertical depth of the well is greater than half the length of the wells major diameter.

10. The inhalation device of claim 1 further comprising a reservoir of powdered inhalant for placing into the well.

11. An inhalation device, comprising:
an air passageway having an inlet end, a narrow portion, and an outlet end, wherein an inlet cross section of the inlet end is larger than an outlet cross section of the outlet end, and a cross section of the narrow portion is smaller than the inlet and outlet cross sections; and
a well having an opening that is disposed along the air passageway at or near the narrow portion and configured to receive a dose of an inhalable powder, the well configured so that a flow of inspired air through the air passageway draws the inhalable powder out of the well and through the inhalation device.

12. The inhalation device of claim 11 wherein the air passageway is a smooth, unitary path through a single-piece housing or is a path through a housing having two portions.

13. The inhalation device of claim 11, wherein a cross section of the well is an ellipse having a long axis in the direction of flow.

14. The inhalation device of claim 11, wherein a center of the well is placed at about 50 to 70% of a distance between the inlet and outlet ends.

15. The inhalation device of claim 11, wherein a cross section of the narrow portion is about 15% to 20% of a cross section of the inlet cross section.

16. The inhalation device of claim 11, further comprising an air flow restriction between the inlet end and the outlet end, the air flow restriction placed at least partly in the narrow portion of the air passageway.

17. The inhalation device of claim 11, wherein a distance from the inlet end to a nearest portion of the well is at least 1.50 times a distance from an opposite end of the well to the outlet end.

18. The inhalation device of claim 11, wherein the well is configured as part of at least one insert of the housing.

19. An inhalation device, comprising:
a housing comprising a smooth, unitary, tapering air passageway having an inlet end, a narrow portion, and an outlet end, wherein an inlet cross section of the inlet end is larger than an outlet cross section of the outlet end, and a cross section of the narrow portion is smaller than the inlet and outlet cross sections; and
a well having an opening that is disposed along the air passageway and configured to receive a dose of an inhalable powder, the well configured so that a flow of inspired air through the air passageway vacuums the inhalable powder out of the well and through the inhalation device.

20. The inhalation device of claim 19, further comprising a cover over at least one of the inlet end, outlet end, and well.

21. The inhalation device of claim 19, wherein an area of the inlet end is at least 1.1 times an area of the outlet end.

22. The inhalation device of claim 19, wherein an area of the inlet end is at least 4 times an area of the narrow portion.

23. The inhalation device of claim 19, wherein the outlet cross section is at least around 190% larger than the cross section of the narrow portion.
